(19) United States
(12) Patent Application Publication
(10) Pub. No.: US 2007/0049648 A1
(43) Pub. Date: Mar. 1, 2007

(54) MANUFACTURE OF FUELS BY A CO-GENERATION CYCLE

(76) Inventor: Gerry Shessel, North York (CA)

Correspondence Address: BERESKIN AND PARR, 40 KING STREET WEST, BOX 401, TORONTO, ON, M5H 3Y2, CA

(21) Appl. No.: 11/467,323

(22) Filed: Aug. 25, 2006

Related U.S. Application Data
(60) Provisional application No. 60/710,879, filed on Aug. 25, 2005.

(51) Int. Cl. C07C 27/06; F02C 1/00

(52) U.S. Cl. 518/702; 60/772

(57) ABSTRACT

A process for the production of a hydrocarbon comprises subjecting a feed stream comprising carbon dioxide and a source of hydrogen to elevated temperatures in an oxygen reduced environment and obtaining a synthesis gas stream comprising carbon monoxide and hydrogen gas; and, utilizing the synthesis gas stream to produce at least one hydrocarbon product.

### FIELD OF THE INVENTION

This invention relates to an integrated, synergistic method of producing power, alcohols, such as methanol, ethanol and higher alcohols, and other higher hydrocarbon compounds, such as carboxylic acids in which a synthesis gas is produced from a hydrocarbon, such as methane and/or carbon dioxide and hydrogen at elevated temperatures in the absence of material amounts of oxygen. Optionally, one or both of the shaft power and the waste heat of an engine may be used to prepare fuels and various hydrocarbon compounds both for use in the engine and for merchant sale. The engine may be used to prepare the fuel using a high efficiency cogeneration cycle and provide incineration of all hydrocarbon byproducts from the process and reduction of greenhouse gases.

### BACKGROUND OF THE INVENTION

Combustion turbine engine cycles are of two types. The simplest type is the single or open cycle type in which a fuel is combusted in a combustion turbine engine wherein the engine produces shaft power that is used to drive a device, such as an electricity generator, and hot exhaust gas that is vented to the atmosphere. No use is made of the available heat energy in the hot exhaust gas from the combustion turbine engine.

The second type of combustion turbine engine cycle available is the combined cycle type in which a fuel is combusted in a combustion turbine engine wherein the engine produces shaft power that is used to drive a device such as an electric generator and hot exhaust gas that is fed to a heat recovery steam generator (HRSG). The HRSG, with or without firing additional fuel, produces steam for use in a steam turbine that produces shaft power and the shaft power is used to drive a device, such as an electric generator. In order to return the steam from the boiler and steam turbine so that it may be pumped up to the boiler pressure and continuously recycled, the steam is condensed to water as it exits the steam turbine. Cooling of the steam is typically provided by indirect heat exchange using water from a local body of water. This is a major source of heat pollution to rivers, lakes and even coastal waters. Cooling of the steam may also be provided by heat exchange to the atmosphere similarly wasting the latent energy in the steam.

The trend in the electrical power industry is to produce power using single or combined cycles, without cogeneration, which causes environmental damage.

Generally stationary combustion turbine engines are operated using natural gas as the fuel, though they are capable of operating on other fuels such as Number 2 fuel oil or other high quality fuels. Other lesser quality fuels are available, such as biogas and landfill gas, produced by the anaerobic digestion of organic materials. Landfill gas is a mixture of approximately equal parts of methane and carbon dioxide with minor quantities of organic and metalorganic compounds present as impurities. In recent years some small quantities of electrical power have been produced using landfill gas as the fuel in reciprocating or combustion turbine engines. These plants have suffered poor economics due to their small size and to the low efficiency of the single cycle compared to plants operating on the combined cycle. They have been prevented from adopting the combined cycle due to the corrosive effects of the impurities in the fuel in the reciprocating or combustion turbine engine and in the heat recovery steam generator. The economics of the electrical power industry do not provide for cleanup of these fuels.

Currently alcohols are produced by a variety of methods, all of which are producers of carbon dioxide, a greenhouse gas. For example, methanol is produced by steam reforming of natural gas, accompanied by the production of carbon dioxide, largely from the fuels used to heat the process. Ethanol may be produced by the fermentation of sugars, with the production of carbon dioxide as a major by product.

McGregor et al. (U.S. Pat. No. 5,416,245) discloses that carbon monoxide may be produced from carbon dioxide by thermal decomposition of the carbon dioxide using the heat produced in a partial oxidation and producer gas process (see for example column 6, lines 40-48). A disadvantage of this process is that no additional hydrogen is produced in the process requiring that additional hydrogen must be imported or manufactured within the plant to affect alcohol manufacture in stoichiometric balance with the available carbon monoxide.

### SUMMARY OF THE INVENTION

In accordance with the instant invention, a synthesis gas is produced at elevated temperatures and in an oxygen reduced environment. At elevated temperatures, the molecules in the feed gas will dissociate and recombine. Preferentially carbon will recombine with oxygen. If the amount of oxygen is controlled, then the amount of oxygen present to combine with hydrogen will be limited and, preferably, essentially no material amount of oxygen will combine with hydrogen. Accordingly, the resultant synthesis gas will contain a reduced amount, and preferably essentially no material amount of water. Advantageously, unlike the use of steam reformation or partial oxidation to produce a synthesis gas, the synthesis gas may be used to prepare one or more alcohols without a preliminary condensation step, or other treatment step, to remove water prior to providing the synthesis gas to a methanol synthesis unit.

In accordance with one aspect of the instant invention, there is provided a process for the production of a hydrocarbon comprising the steps of:

(a) providing a gaseous feed stream comprising carbon dioxide and a source of hydrogen;

(b) subjecting the feed stream to elevated temperatures in an oxygen reduced environment and obtaining a synthesis gas stream comprising carbon monoxide and hydrogen gas, and,

(c) utilizing the synthesis gas stream to produce at least one hydrocarbon product.

In one embodiment, the hydrogen is obtained from a hydrocarbon. Preferably the hydrocarbon comprises methane. Preferably, the feed stream comprises approximately equal amounts of methane and carbon dioxide on a mole basis.

In another embodiment, the hydrocarbon product comprises at least one alcohol. Preferably, the at least one alcohol comprises ethanol.

In another embodiment, the carbon dioxide is obtained from one or more of biogas, landfill gas, natural gas, beverage alcohol production, gasification of hydrocarbons and the combustion of coal.

In another embodiment, the elevated temperature is from 1,110° C.-3,200° C.

In another embodiment, the elevated temperature is from 1,700° C.-2,300° C.

In another embodiment, the elevated temperature is about 2,200° C.

In another embodiment, the oxygen reduced environment comprises less than 10 weight percent oxygen gas, and preferably less than 5 weight percent oxygen gas. In one preferred embodiment, the reforming reactor is not provided with a supply of oxygen gas (e.g., there is no air stream directed to the reactor). Accordingly, step (b) is conducted in the absence of the addition of combustion air or oxygen.

It will be appreciated that air or oxygen may be entrained in the fuel supplied to the process, e.g. as in coal.

In another embodiment, the oxygen reduced environment comprises essentially no oxygen gas,

In another embodiment, step (b) comprises a dry reforming process.

In another embodiment, step (b) comprises a multi-stage dry reforming process.

In another embodiment, the process further comprises utilizing combustion of a fuel to produce electricity and hot combustion gasses and utilizing at least one of a portion of the electricity and at least a portion of the hot combustion gasses to provide heat to the dry reforming step.

In another embodiment, the process further comprises utilizing excess heat available in a plant to provide heat to the dry reforming step and to produce steam and utilizing at least a portion of the steam to produce electricity.

In another embodiment, the process further comprises treating coal to obtain carbon dioxide, which is utilized to produce the feed gas.

In another embodiment, the process further comprises treating coal to obtain methane which is utilized to produce carbon dioxide.

Pursuant to a particularly preferred embodiment of the instant invention there is also provided a process for the production of power, alcohols and hydrocarbon compounds comprising the steps of:
a. preparing a feed stream including a mixture of a gaseous organic combustible fuel and carbon dioxide from available individual or collective sources, for example: biogas, landfill gas, natural gas, beverage alcohol production, gasification of carbohydrates, combustion of coal, etc,
b. feeding said feed stream, formed in step a, to the first stage of a gas reformer unit wherein the mixture of fuel and carbon dioxide in said feed stream absorbs heat from the stream of low pressure steam exiting from a steam turbine, as in step p, thereby simultaneously heating the mixture of fuel and carbon dioxide in said feed stream and condensing the steam exiting the steam turbine,
c. further feeding said heated feed stream to the second stage of a gas reformer unit wherein said heated feed stream absorbs additional heat from the hot gas stream exiting from stage four of the gas reformer, as in step g, thereby simultaneously heating, either in the presence or in the absence of a catalyst, the mixture of fuel and carbon dioxide in said feed stream causing the mixture of fuel and carbon dioxide in said feed stream to shift the equilibrium of its composition toward a mixture of fuel, carbon dioxide, carbon monoxide and hydrogen and cooling the hot gas stream from stage four of the gas reformer,
d. further feeding said heated feed stream to the third stage of a gas reformer unit wherein said heated feed stream absorbs additional heat from the hot exhaust gas stream from a reciprocating or combustion turbine engine, formed in steps k and l, thereby simultaneously heating, either in the presence or in the absence of a catalyst, the fuel, carbon dioxide, carbon monoxide and hydrogen mixture in said feed stream causing the mixture of fuel, carbon dioxide, carbon monoxide and hydrogen to further shift the equilibrium of its composition toward a mixture of carbon monoxide and hydrogen with lesser amounts of the fuel and carbon dioxide and cooling the hot exhaust gas stream from the reciprocating or combustion turbine engine,
e. further feeding said heated feed stream to the fourth stage of a gas reformer unit wherein said heated feed stream further absorbs heat from a hot gas stream exiting from stage five of the gas reformer, as in step g, thereby simultaneously heating, either in the presence or in the absence of a catalyst, the mixture of fuel, carbon dioxide, carbon monoxide and hydrogen in said heated feed stream causing the mixture of fuel, carbon dioxide, carbon monoxide and hydrogen to shift the equilibrium of its composition toward a mixture of carbon monoxide and hydrogen with lesser amounts of the fuel and carbon dioxide and cooling the hot gas stream from stage five of the gas reformer,
f. further feeding said heated feed stream to the fifth stage of a gas reformer unit wherein said heated feed stream absorbs further heat from electrical immersion heaters supplied with electricity, as in step r, thereby simultaneously heating, either in the presence or in the absence of a catalyst, the mixture of fuel, carbon dioxide, carbon monoxide and hydrogen in said heated feed stream causing the mixture of fuel, carbon dioxide, carbon monoxide and hydrogen to further shift the equilibrium of its composition toward a mixture of carbon monoxide and hydrogen with lesser amounts of the fuel and carbon dioxide,
g, cooling the heated feed stream of carbon monoxide and hydrogen with lesser amounts of the fuel and carbon dioxide produced in steps b, c, d, e and f by utilizing it as the heat source in steps c and e thereby cooling said stream to prevent the reverse shift of the equilibrium of its composition,
h. performing separations of the gases carbon monoxide and hydrogen to achieve the stoichiometrically correct feed stream composition to perform conversions of these to alcohols and other hydrocarbon compounds,
i. feeding at least a portion of said adjusted stream to one or more of a variety of alcohol and other hydrocarbon compound conversion processes to form alcohols and other hydrocarbon compounds,
j. performing product separations to recover saleable alcohol and other hydrocarbon compound products for merchant sale,
k. feeding at least a portion of the carbon monoxide and hydrogen surplus from step h as a portion of the fuel to a reciprocating or combustion turbine engine producing shaft power and hot exhaust gas,
l. raising the temperature of, and the heat content in, the hot exhaust gas stream from the reciprocating or combustion turbine engine, produced in step k, by feeding at least a portion of the carbon monoxide and hydrogen surplus from step h as a portion of the fuel combusted in the surplus oxygen in the stream of hot exhaust gas,
m. feeding the exhaust gas stream from the reciprocating or combustion turbine engine, produced in steps k and l, to stage three of the reformer, as in step d,
n. further feeding the exhaust gas stream from the reciprocating or combustion turbine engine, as in step m, to a heat recovery steam boiler to produce a stream of high pressure steam,
o. expanding the stream of high pressure steam, produced in step n, through a steam turbine to produce shaft power and low pressure steam,
p. feeding the low pressure steam, produced in step o, to stage 1 of the reformer, as in step b, thereby simultaneously heating the fuel and carbon dioxide mixture in the feed stream and condensing the steam to water,
q. raising the pressure of the water produced in step p to boiler pressure for recirculation,
r. utilizing at least a portion of the shaft power produced in steps k and o to produce electric power to supply at least a portion of the electric power to power the electric immersion heaters used in step f.

As discussed herein, it will be appreciated that all of these steps may not be utilized.

### BRIEF DESCRIPTION OF THE DRAWINGS

These and other advantages of the instant invention may be more completely and fully understood by means of the following description of the accompanying drawings of the preferred embodiments of this invention in which:

FIG. 1 is a schematic of a process flow sheet of one embodiment of this invention in which a mixture of a gaseous organic combustible fuel and carbon dioxide is reformed to carbon monoxide and hydrogen to prepare these as precursors for production of alcohols and other hydrocarbon compounds.

FIG. 2 is a schematic of a process flow sheet of a second embodiment of this invention in which a mixture of a gaseous organic combustible fuel and carbon dioxide is reformed to carbon monoxide and hydrogen and in which the carbon monoxide and hydrogen is used to produce alcohol.

FIG. 3 is a schematic of a process flow sheet of a third embodiment of this invention in which a mixture of a gaseous organic combustible fuel and carbon dioxide is reformed to carbon monoxide and hydrogen and in which the carbon monoxide and hydrogen is used to produce methanol followed by the carbonylation or homologation of the methanol to form higher alcohols and/or other hydrocarbon compounds.

FIG. 4 is a schematic of a process flow sheet of a fourth embodiment of this invention in which a mixture of a gaseous organic combustible fuel and carbon dioxide is reformed to carbon monoxide and hydrogen and in which alcohol and other hydrocarbon compounds is achieved by the fermentation of the carbon monoxide and hydrogen by microbial catalyst.

FIG. 5 is a schematic of a process flow sheet of a fifth embodiment of this invention in which the production of alcohols is achieved by the microbial digestion of organic materials to form a mixture of the gases methane and carbon dioxide followed by reforming of these to form the carbon monoxide and hydrogen followed by the fermentation of the carbon monoxide and hydrogen to alcohols and other hydrocarbons by microbial catalyst.

FIG. 6 is a schematic of a process flow sheet of a sixth embodiment of this invention in which the production of alcohols is achieved by the combustion of organic materials to form carbon dioxide followed by reforming of the carbon dioxide with methane to form carbon monoxide and hydrogen followed by the fermentation of the carbon monoxide and hydrogen to alcohols and other hydrocarbons by microbial catalyst.

FIG. 7 is a schematic of a process flow sheet of a seventh embodiment of this invention in which the production of alcohols is achieved by the fermentation of carbon monoxide by microbial catalyst to alcohols, carbon dioxide and other hydrocarbons followed by reforming of the carbon dioxide with methane to form carbon monoxide which is recycled in the process.

### DETAILED OF THE PREFERRED EMBODIMENT

According to the instant invention a synthesis gas is obtained by subjecting a feed gas to elevated temperatures, and optionally elevated temperatures and pressures, in an oxygen reduced environment and, preferably, in the absence of oxygen. At elevated temperatures, hydrocarbon compounds will tend to dissociate into their constituents, e.g., carbon atoms, hydrogen atoms and oxygen atoms. When carbon dioxide is heated in the presence of a hydrocarbon compound the carbon atoms tend to seek out oxygen atoms more aggressively than do the hydrogen atoms. By limiting the amount of oxygen that is present, the carbon atoms will preferentially combine with the oxygen atoms resulting in the hydrogen atoms not being able to combine with oxygen atoms (which would produce water) and, accordingly, the hydrogen atoms will combine with other hydrogen atoms to produce hydrogen gas. For example, if the chosen hydrocarbon compound is methane, a fuel gas which is commonly produced together with carbon dioxide in biological processes, then a carbon monoxide and hydrogen mixture may be prepared by heating a stoichiometric mixture of carbon dioxide and methane using the hot gas discharged from the turbine, according to the reaction.
CH₄+CO₂→2CO+2H₂

This is an equilibrium reaction. This reaction, which may be referred to as dry reforming, may be distinguished from steam reformation and partial oxidation reactions in which additional oxygen atoms are provided to alter the composition of the resultant synthesis gas. In a steam reformation process, water molecules, in the form of steam, are added to the feed gas, thereby supplying additional hydrogen atoms and oxygen atoms to the feed gas. Steam reformation may be utilized if the feed gas does not contain a desired amount of hydrogen atoms to produce a synthesis gas of the desired composition. In a partial oxidation process, oxygen atoms (e.g., by providing air or oxygen enriched air to the feed gas) are provided to the feed gas.

The feed gas may be the gas or vapor phases of any of the homologues of the families of alkanes, alkenes and alkynes. Such gasses may be obtained naturally, for example from the off gasses from landfill sites, natural gas, anaerobic digestion of animal or vegetable materials or artificially manufactured by the hydrogenation of carbon as explained below.

The dry reforming reaction is endothermic and is promoted by high temperatures. The dry reforming reaction may be conducted at 1,100° C.-3,200° C., preferably 1,700° C.-2,300° C. and more preferably about 2,200° C.

The reaction is preferably conducted in the absence of, or essentially in the absence of, oxygen gas. Accordingly, if a feed gas has a significant amount of oxygen present, e.g. an amount that will materially alter the composition of the synthesis gas produced by the dry reforming, then some or all of the oxygen may be removed by, e.g., gas separation techniques, by condensation and/or by passing the feed gas through iron wool filter media. However, in many cases, the feed gas may be obtained from an industrial process gas (e.g. anaerobic digestion, carbonization, etc.), which does not contain oxygen.

One preferred feed gas that may be utilized is landfill gas since it is naturally produced in the desirable ratios, escapes to the environment contributing the global warming and provides a poor source of fuel for engine and generator sets due to its low heating value and entrained inert solids content.

The process is preferably heated using the exhaust gas from a combustion turbine. The temperature of combustion turbine exhaust gases may approach or achieve the temperature required for dry reforming. Accordingly, if combustion turbine exhaust gases are utilized, the feed gas may not require any heating or, only a minimal amount of heating. If the turbine exhaust gas temperature is not sufficiently high to achieve the optimum rate of the reaction then additional fuel, out of the variety of fuels available, may be burned in the exhaust gas stream to elevate its temperature further. This is possible because the surplus of oxygen available in the turbine exhaust gas stream permits the combustion of additional fuels. Additional heating may also be achieved by the use of electric heaters submerged in the gas stream.

This reaction is used to prepare the carbon monoxide and hydrogen, which may then be used as precursors or a synthesis gas for use in the manufacture of alcohols and other higher hydrocarbons,

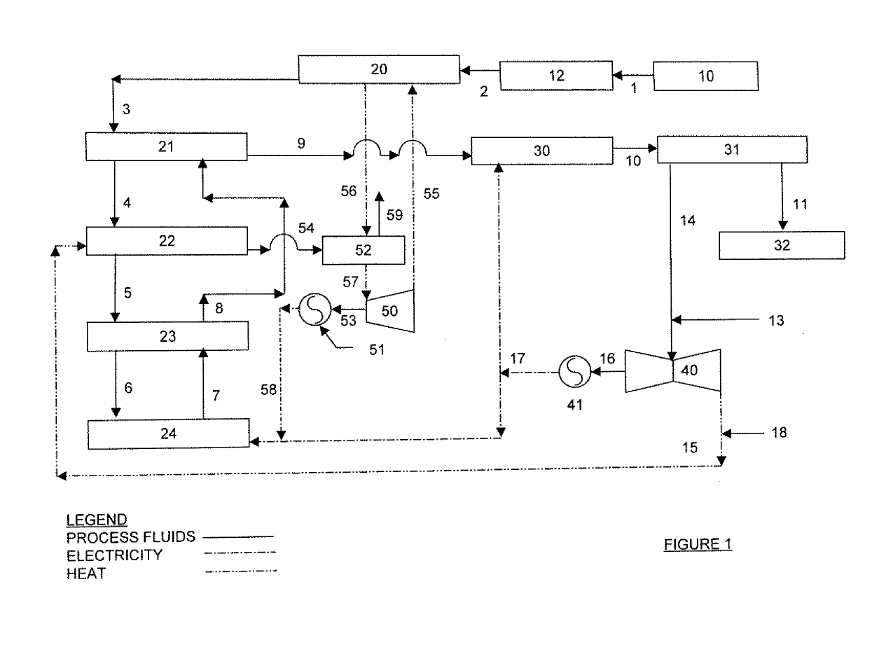

According to the first preferred embodiment, as exemplified in FIG. 1, the process comprises gas source 10, gas cleaning unit 12 multiple stages of the gas reforming unit: stage 1 of the gas reforming unit 20, stage 2 of the gas reforming unit 21, stage 3 of the gas reforming unit 22, stage 4 of the gas reforming unit 23 and stage 5 of the gas reforming unit 24, gas compression unit 30, chemical manufacturing unit 31, reciprocating or combustion turbine engine 40, electrical generator 41, steam turbine 50, electrical generator 51 and heat recovery steam boiler (HRSG) 52.

A feed gas is provided to the process from a gas source 10. Gas source 10 may be one or more storage tanks. As shown in FIG. 1, a mixture of approximately equal parts of a gaseous organic combustible fuel, for example, methane, and carbon dioxide gas is prepared from one or more suitable sources and introduced into the process forming via feed stream 1. The feed stream gas mixture, stream 1, is then preferably treated in gas cleaning unit 12 to remove the contaminants which may injure subsequent components or the quality of the finished products. The cleaned gas is then fed to the gas reformer via stream 2.

The gas reformer may comprise one or more stages. As exemplified in FIG. 1, five stages are utilized. However, it will be appreciated that the number of stages that are utilized will vary depending upon the constituents of cleaned feed gas stream 2, the temperature of cleaned feed gas stream 2, the desired degree of conversion and availability of energy sources and sinks within the plant.

Stage 1 of the gas reforming unit 20 is essentially an indirect counter flow heat exchanger wherein the feed stream mixture of the gases methane and carbon dioxide, cleaned feed gas stream 2, is heated, such as by the low pressure steam in stream 55 thereby forming stream 3. Stage 2 of the gas reforming unit 21 is essentially an indirect counter flow heat exchanger wherein the mixture of the gases methane and carbon dioxide, stream 3, is further heated by the hot reformed gas mixture, stream 8, either in the presence or in the absence of a catalyst, causing the mixture of methane and carbon dioxide to shift the equilibrium of its composition toward a mixture of methane, carbon dioxide, carbon monoxide and hydrogen according to the endothermic process;
CH₄+CO₂→2CO+2H₂,
thereby forming stream 4.

Stage 3 of the gas reforming unit 22 is essentially an indirect counter flow heat exchanger wherein the mixture of the gases methane, carbon dioxide, carbon monoxide and hydrogen, stream 4, is further heated by the hot exhaust gas, stream 15, from a reciprocating or combustion turbine engine 40, either in the presence or in the absence of a catalyst, causing the mixture of methane, carbon dioxide, carbon monoxide and hydrogen to further shift the equilibrium of its composition toward a mixture of carbon monoxide and hydrogen with reduced amounts of methane and carbon dioxide thereby forming stream 5.

Stage 4 of the gas reforming unit 23 is essentially an indirect counter flow heat exchanger wherein the mixture of the gases methane, carbon dioxide, carbon monoxide and hydrogen, stream 5, is heated by the hot reformed gas mixture, stream 7, either in the presence or in the absence of a catalyst, causing the mixture of the gases methane, carbon dioxide, carbon monoxide and hydrogen to further shift the equilibrium of its composition toward a mixture of carbon monoxide and hydrogen with reduced amounts of methane and carbon dioxide thereby forming stream 6.

Stage 5 of the gas reforming unit 24 is essentially an electrical immersion heater wherein the mixture of the gases methane, carbon dioxide, carbon monoxide and hydrogen, stream 6, is heated by electricity, reference numerals 17 and 58, either in the presence or in the absence of a catalyst, causing the mixture of methane, carbon dioxide, carbon monoxide and hydrogen to further shift the equilibrium of its composition toward a mixture of carbon monoxide and hydrogen with reduced amounts of methane and carbon dioxide thereby forming stream 7.

The steam, stream 55, used to heat the cleaned gas stream 2 is preferably produced from the heat inherent in the exhaust gasses produced by combustion, such as the combustion of a fuel to produce power via, e.g. a reciprocating or combustion turbine engine 40. It will be appreciated that other types of engines might be utilized and other sources of gasses may be used. The heat may be used to heat gas reforming stage 1 by any means known in the art, but is preferably conducted by indirect heat exchange, wherein the combustion gases are fed directly to stage 1 or, alternately, are first used in another process, such as another stage in the gas reforming process or in another process in a plant, such as heating of the media in the anaerobic digestion or fermentation processes described below.

Referring to FIG. 1, stream 54 is produced by further cooling of exhaust gas stream 15 from the reciprocating or combustion turbine engine 40, which has previously been cooled in stage 3 of the gas reforming unit thereby forming stream 54. The exhaust gas, stream 54, is further cooled, prior to venting to the atmosphere via stream 59, by heat exchange to the condensate, stream 56, in the heat recovery steam generator (HRSG) 52 producing high pressure steam forming stream 57. The high-pressure steam, stream 57, is expanded through a steam turbine 50 producing shaft power 53 and low-pressure steam stream 55. The low-pressure steam, stream 55, is fed to stage 1 of the gas reforming unit 20 where simultaneously cleaned feed stream 2 is heated forming stream 3 and the low pressure steam, stream 55, is cooled and condensed to water forming stream 56. The condensate, stream 56, is pumped up to pressure and returned to the heat recovery steam generator (HRSG) 52 for heating to again form steam as in stream 57. The shaft power, stream 53, is preferably used to power an electrical generator 51 to convert the mechanical shaft power 53, to electricity 58.

By utilizing a plurality of stages in the gas reformer, stream 2 is progressively heated, reformed to a mixture of carbon monoxide and hydrogen with minor amounts of methane and carbon dioxide and cooled by a series of gas reforming steps, thereby forming synthesis gas stream 9. Preferably, the feed gas to the gas reformer comprises approximately equal parts of methane and carbon dioxide. However, the reformer may utilize methane and carbon dioxide in a ratio of 3:1 to 1/3:1, and preferably about 1:1.

Other gas reforming unit configurations, with greater or lesser numbers of stages of the gas reforming unit, may be advantageous depending on the availability of heat sources and sinks within the plant.

Typically, it is preferred to operate processes that utilize synthesis gas stream 9 at an elevated pressure. Accordingly, a gas compression unit 30 is preferably used to raise the pressure of stream 9 in preparation for subsequent gas separation and conversion processes, thereby forming pressurized stream 10,

Preferably, the synthesis gas is utilized to manufacture more complex hydrocarbons. Accordingly, a chemical manufacturing unit 31 may be used to manufacture chemicals utilizing one or more of a variety of processes, which include, without limitation, synthesis, carbonylation, homologation and/or fermentation, which will convert the precursor, namely the pressurized synthesis gas stream 10 that may contain carbon monoxide and hydrogen, into alcohols, including methanol, ethanol and higher alcohols and other higher hydrocarbon compounds forming product stream 11. The product stream may be stored in one or more storage tanks 32 for later use or sale. The chemical manufacturing unit 31 preferably includes methods of separating and refining these products in preparation for use and/or merchant sale. Any such techniques known in the art may be used.

A fuel is prepared and fed, via stream 13, to the reciprocating or combustion turbine engine 40. A reciprocating or combustion turbine engine 40, upon burning the fuel, produces (1) shaft power 16 and (2) rejected heat in the coolant fluids and exhaust gas forming stream 15. The shaft power 16 is preferably used to power an electrical generator 41 to convert the mechanical shaft power 16 to electricity 17, which may be used or sold.

Heat is available in a broad range of qualities. Reciprocating engines, due to limitations of metallurgy and lubrication, reject heat at low temperature, deemed low quality heat. Combustion turbine engines, free of these limitations, reject heat at high temperature, deemed high quality heat. Higher temperatures of and greater heat contents in the exhaust gas, stream 15, can be achieved by burning additional fuels, stream 18, in the remaining oxygen in the exhaust gas, stream 15.

In operation, each of the variety of methods of chemical manufacture 31 do not completely convert the precursors, carbon monoxide and hydrogen, to alcohols and other higher hydrocarbon compounds leaving quantities of either or both surplus forming stream 14. Both carbon monoxide and hydrogen are suitable for use as fuels. Therefore the surplus carbon monoxide and hydrogen are preferably fed, such as via stream 14, to fuel the reciprocating or combustion turbine engine reducing or eliminating the requirement for imported fuel, stream 13.

The hot exhaust gas mixture from the reciprocating or combustion turbine engine 40, stream 15 is preferably used for heating the gas mixture, stream 4, in stage 3 of the gas reforming unit 22. The electricity produced, 17, is preferably used within the plant for gas compression, for additional heating of the gas mixture, stream 6, in stage 5 of the gas reforming unit 24 using immersion heaters in the gas stream, and/or it is exported from the plant for merchant sale.

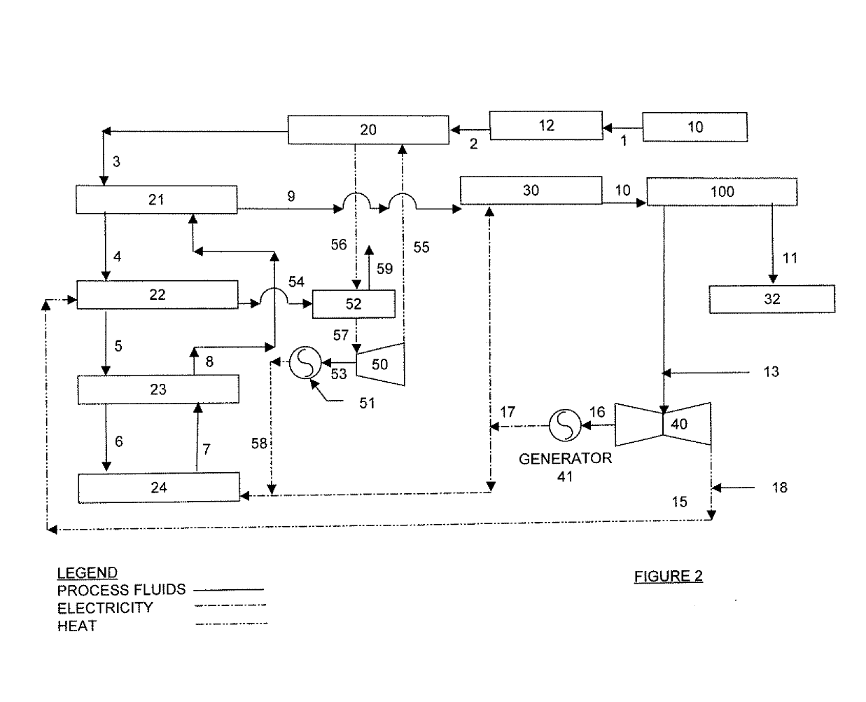

The second preferred embodiment, as exemplified in FIG. 2, is similar to the preferred embodiment of FIG. 1 except that in place of a chemical manufacturing unit 31, an alcohol synthesis and separations unit 100 is used to convert the precursors, carbon monoxide and hydrogen, stream 1 into one or more alcohols, such as methanol, ethanol and higher alcohols. The alcohol synthesis and separations unit 100 includes methods of separating and refining these products in preparation for merchant sale. Alcohols and other higher hydrocarbon compounds may be prepared from the precursors carbon monoxide and hydrogen by synthesis over a variety of catalysts at elevated temperatures and pressures. Product stream 11 will typically be a suite of product alcohols including methanol, ethanol and higher alcohols and other higher hydrocarbon compounds, which are available for merchant sale.

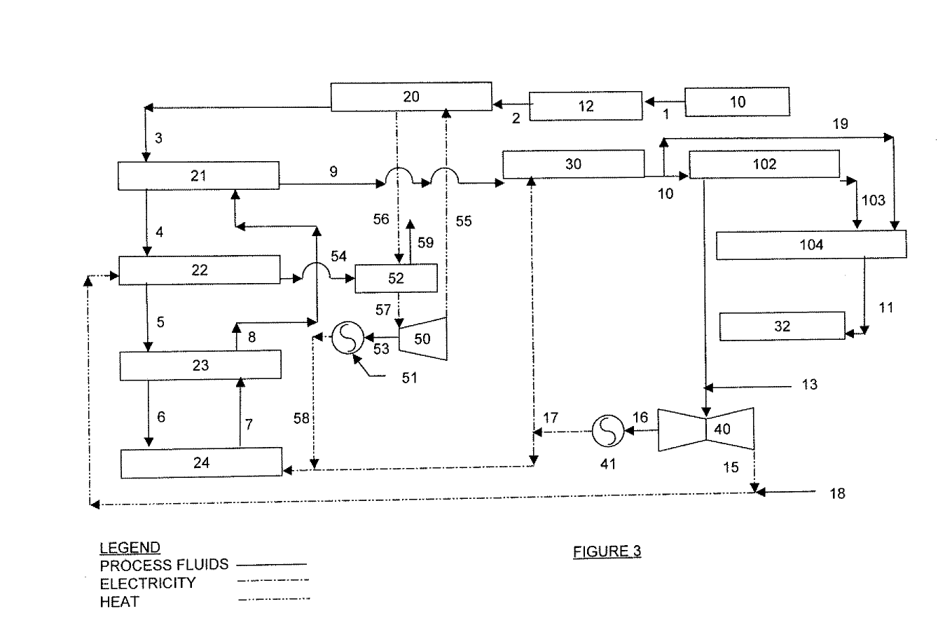

The third preferred embodiment, as exemplified in FIG. 3, is similar to the preferred embodiment of FIG. 1 except that in place of a chemical manufacturing unit 31, a methanol synthesis and separations unit 102 converts the precursors, carbon monoxide and hydrogen, stream 10, into methanol stream 103 The methanol synthesis and separations unit 102 includes methods of separating and refining the methanol in preparation for use or further processing. The methanol may then be converted to higher alcohols by homologation or by carbonylation. The balance of the material in pressurized stream 10, stream 19, is diverted to the homologation and separations unit 104 or to the carbonylation and separations unit 104 where it is combined with stream 103, the methanol formed in the methanol synthesis and separations unit 102. Methanol, such as from stream 103, may be homologated with additional carbon monoxide and hydrogen, such as stream 19, into alcohols and other higher hydrocarbon compounds including ethanol and higher alcohols in the homologation and separations unit 104, see for example, Leung, et al. (U.S. Pat. No. 4,935,547). The alcohol homologation and separations unit 104 preferably includes methods of separating and refining these products in preparation for merchant sale. Methanol, such as stream 103, may be carbonylated with additional carbon monoxide and hydrogen, such as stream 19, into alcohols and other higher hydrocarbon compounds including ethanol and higher alcohols in the carbonylation and separations unit 104, see for example Gauthier-Lafaya, et al. (U.S. Pat. No. 4,306,091). The alcohol carbonylation and separations unit 104 includes preferably methods of separating and refining these products in preparation for merchant sale. Product stream 11 is the suite of product alcohols and other higher hydrocarbon compounds including methanol, ethanol and higher alcohols, which are available for merchant sale.

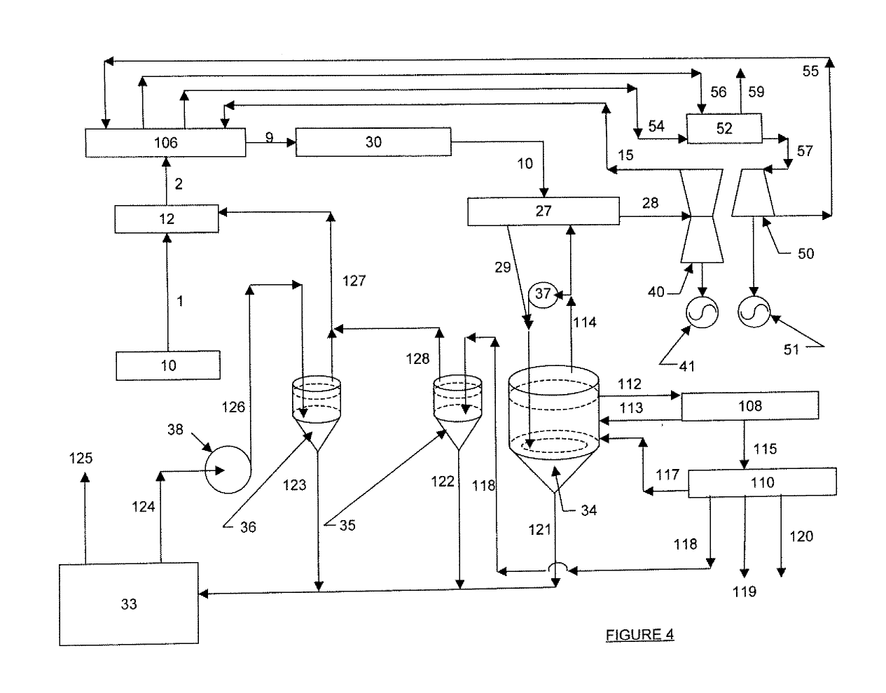

The fourth preferred embodiment, as exemplified in FIG. 4, exemplifies the conversion of landfill gas, such as may be collected from a landfill gas source 10, to produce ethanol. In order to simplify the diagram, the multistage reformer is designated by reference numeral 106. In addition, the process utilizes active solids separator unit 108, product separator unit 110, inactive solids separator unit 33, alcohol fermentation reactor unit 34, hydrocarbon compounds digestion reactor unit 35, digestion reactor unit 36, blower 37, and organic material slurry pump 38.

A mixture, preferably of approximately equal parts of methane and carbon dioxide gas is prepared from one or more landfills, organic waste digesters, sewage treatment plants and other similar processes. The feed gas stream 1 is preferably treated as described with respect to FIG. 1 to obtain a pressurized synthesis gas stream 10, which is optionally separated in the gas separation unit 27 to obtain two, or more gas streams as may be required for further processes. As exemplified in FIG. 4, gas separation unit 27 is utilized to:
1. separate at least a portion of the hydrogen from the mixed pressurized gas stream 10 that is used as a fuel in the reciprocating or combustion turbine engine 40, thereby forming stream 28,
2. separate at least a portion of the carbon monoxide from the mixed pressurized gas stream 10 for use as a feed stream to the alcohol fermentation reactor unit 34, thereby forming stream 29.

Carbon monoxide or mixed carbon monoxide and hydrogen, stream 29, is used as a substrate for the growth and support of the microbial catalyst in, e.g., the alcohol fermentation reactor unit 34. The feed stream of carbon monoxide or mixed carbon monoxide and hydrogen, stream 29, is converted to alcohol or mixed alcohols and other higher hydrocarbon compounds in the alcohol fermentation reactor unit 34. The alcohol fermentation reactor unit 34 typically contains an aqueous media containing vitamins, metals, minerals and nutrients and a suspension of microbial catalyst. Known microbial catalysts which convert carbon monoxide or mixed carbon monoxide and hydrogen to alcohols and other higher hydrocarbon compounds include without limitation:
1. Acetobacterium woodii and various strains thereof,
2. Butyribacterium methylotrophicum and various strains thereof,
3. Clostridium acetobutylicum and various strains thereof,
4. Clostridium formicaceticum and various strains thereof,
5. Clostridium thermoaceticum and various strains thereof,
6. Clostridium thermoautotrophicum and various strains thereof,
7. Essherichia coli and various strains thereof,
8. Thermoanaerobacter ethanolicus and various strains thereof,
9. Thermoanaerobacter thermohydrosulfuricus and various strains thereof.

The alcohol fermentation reactor 34 is preferably provided in two stages. The first stage provides for the growth of the microbial catalyst on the gas substrate with the media pH suitably adjusted. The second stage provides for the production of mixed alcohols on the gas substrate with the media pH suitably adjusted. Alternatively the reactor may contain a liquid, for example, perflourocarbon, ethanolamine, etc. and/or aqueous solutions and/or emulsions of these, in which the carbon oxides and/or hydrogen gases are soluble. The reactor is provided with spargers, with agitators, with pH controllers and with temperature control for heating and cooling of the contents. Stream 114 is the mixture of carbon monoxide and carbon dioxide or the mixture of carbon monoxide, carbon dioxide and hydrogen, which remains unconverted in the alcohol fermentation reactor unit 34. Stream 114 may optionally be:
1. returned directly to the alcohol fermentation reactor unit 34 for further conversion of the carbon oxides and/or hydrogen to mixed alcohols,
2. returned to the gas separation unit 27 for separation of the mixed gases,
3. fed sequentially to a series of alcohol fermentation reactor units 34 so that the carbon oxides are progressively converted to alcohols leaving substantially only carbon dioxide remaining in the gas stream, and/or
4. vented to the air intake of the gas turbine 40 for incineration and disposal of organic contaminants.

The blower 37 is preferably used to raise the gas pressure in stream 114 to provide sufficient pressure in the gas stream 114 to allow reintroduction of the gas stream 114 into the base of the liquid in alcohol fermentation reactor unit 34.

A stream of media containing the live microbial catalyst in suspension and mixed alcohols and other higher hydrocarbon compounds in solution is removed from the alcohol fermentation reactor unit 34 forming raw product stream 112, which is treated to recover alcohols and other higher hydrocarbon compounds which may be present. For example, an active solids separator unit 108 may be used to concentrate the suspended live microbial catalyst in a portion of the media and recycles this portion back to the alcohol fermentation reactor 34 via stream 113. The balance of the stream of media solution deficient in microbial catalyst is directed to the product separation unit 110 via stream 115. The product separator unit 110 separates the alcohols and other higher hydrocarbon compounds from the stream of media solution 115, for refining and export from the plant such as via streams 117, 118, 119 and 120.

Stream 117 is the stream of media stripped of alcohols and other higher hydrocarbon compounds, which is preferably recycled to the alcohol fermentation reactor unit 34. Stream 118 is the stream of higher hydrocarbon compounds produced in the alcohol fermentation reactor unit 34 to be refined and exported from the plant or directed to the hydrocarbon compounds digestion reactor unit 35 for further processing. Stream 119 is the stream of an alcohol, for example, ethanol, produced in the alcohol fermentation reactor unit 34 to be refined and exported from the plant. Stream 120 is the stream of an alcohol, for example butanol, produced in the alcohol fermentation reactor to be refined and exported from the plant.

Waste organic solids, for example, spent microbial catalyst, are formed in the alcohol fermentation reactor unit 34. These are preferably delivered to the inactive solids separator unit 33 for solids separation via stream 121, so that the media and the solids are recycled separately to the plant. Waste organic solids, for example, spent microbial catalyst, may also be formed in the hydrocarbon compounds digestion reactor unit 35. These may also be delivered to the inactive solids separator unit 33 for solids separation via stream 122. Waste organic solids, for example, spent microbial catalyst, may also be formed in the digestion reactor unit 36. These may also be delivered to the inactive solids separator 33 for solids separation thereby forming stream 123.

The inactive solids separator unit 33 preferably concentrates the solids in a portion of the media to form a slurry and returns the solids to the process for destruction via stream 124. The balance of the media is preferably delivered to the media treatment/water treatment plant for recovery and recycling via stream 125. The organic material slurry pump 38 transports the organic material slurry stream 124 to digestion reactor unit 36 via stream 126.

The digestion reactor 36 preferably digests the organic materials in an approximately 10% to 15% by weight suspension of finely divided organic solids in aqueous media, anaerobically, in a three stage process. The first stage is depolymerization of the organic matter by enzymatic hydrolysis to organic acids. The second stage is the decomposition of these organic acids by acetogenic bacteria to acetic acid, hydrogen gas, nitrogen gas and carbon dioxide gas. The third stage is the reforming of the acetic acid, hydrogen gas and carbon dioxide gas by one of a variety of methanogenic bacteria to produce 50% to 70% by weight methane and 30% to 50% by weight carbon dioxide gas. The reactor may be provided with agitators, with pH controllers and with temperature control for heating and cooling of the contents. Stream 127 contains the products of anaerobic digestion, substantially methane and carbon dioxide gases, saturated with water vapor, which is preferably recycled into the process thus avoiding creation of waste materials from the process.

The hydrocarbon compounds digestion reactor unit 35 preferably digests the feed stream of higher hydrocarbon compounds, stream 118, anaerobic ally by one or more of a variety of methanogenic bacteria to carbon dioxide and methane, thereby forming stream 128. Known microbial catalysts, which reduce higher hydrocarbon compounds, include without limitation Acetobacterium woodii and various strains thereof.

The higher hydrocarbon compounds reactor unit 35 may be provided with agitators, with pH controllers and with temperature control for heating and cooling of the contents. Stream 28, similar to stream 12 in FIG. 1, is the stream of fuel gas containing at least one of hydrogen and carbon monoxide which may be used to produce power and heat.

The balance of the plant is similar to the preferred embodiment of FIG. 1 and as described in the forgoing. The plant produces large quantities of low quality heat in the low pressure steam, stream 55, in the condensate, stream 56 and in the exhaust gas, stream 59 which may be used in the plant for process or space heating. The uses of steam or condensate include without limitation:
1. Reactor and tank heating within the plant,
2. Building heating;
3. Greenhouse heating,
4. Aquaculture heating,
5. Preparation of boiler feed water (BFW) and media.

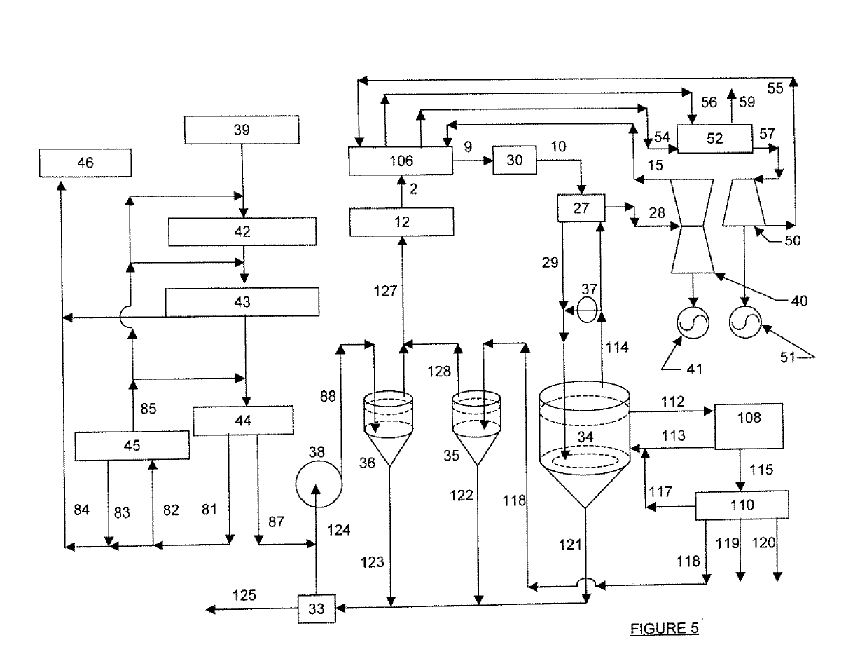

The fifth preferred embodiment, as exemplified in FIG. 5, exemplifies the conversion of municipal solid waste to produce, e.g., ethanol the process is similar to that of FIG. 4 but also comprises a receiving hopper 39, hammer mill 42, tramp metal detector/separator 43, deboning machine 44, refuse classifier 45, refuse hopper 46,

The plant receives mixed municipal solid waste and treats the waste to produce a feed suitable for a digestion reactor 36. Alternately, the plant may obtain such a feedstock that has already been obtained from municipal solid waste. The feedstock may be obtained by any means known in the art. For example, as exemplified in FIG. 5, organic, inorganic substrate and refuse materials may be introduced into the process through the receiving hopper 39. The hammer mill 42 tears open plastic bags and reduces received materials to the inlet opening size of the deboning machine 44. The tramp metal detector/separator 43 protects the deboning machine 44 from damage due to tramp metals in the received material. Tramp metal is separated from the received material and directed to the refuse hopper 46. The deboning machine 44 separates the organic materials from inorganic substrates, for example, bones, and tramp materials, for example, plastic bags, in the received material. A deboning machine 44 is typically an enclosed space, for example, a tube, in which the walls are provided with:
1. a large entry opening suitable for the ingress of mixed organic, inorganic substrate and refuse materials,
2. a large number of small openings, for example holes or slots, through which organic materials may exit in finely divided form, and
3. a large exit opening suitable for the discharge of inorganic substrate and refuse materials.

The mixed organic, inorganic substrate and refuse materials are introduced into the enclosed space through the entry opening. The materials within the enclosed space are placed under pressure, by for example, a screw or ram. The materials are separated when the organic materials exit through the small openings and the inorganic substrate and refuse materials are retained within the enclosed space. The inorganic substrate and refuse materials are discharged from the enclosed space through the exit opening. Stream 81 is the stream of essentially mixed inorganic substrate and refuse materials, with some organic materials possibly entrained within, which has been removed from the process. Stream 81, may be directed either to the refuse classifier 45 via stream 82 or the refuse hopper 46 via stream 84 for removal from the plant.

When there is some organic material entrained within the inorganic substrate and refuse materials the mixed organic, inorganic substrate and refuse materials are preferably directed via stream 82 to the refuse classifier 45. The refuse classifier 45 separates the organic material entrained within the inorganic substrate and refuse materials. When there is no organic material entrained within the inorganic substrate and refuse materials, the mixed inorganic substrate and refuse materials are preferably directed via stream 83 to the refuse hopper 46. When there is organic material entrained within the inorganic substrate and refuse materials, the mixed organic materials are separated from the inorganic substrate and refuse materials in the refuse classifier 45. The inorganic substrate and refuse materials are directed via stream 83 to the refuse hopper 46. The organic material is preferably recycled via stream 85 to the hammer mill, the tramp metal detector/separator and/or the deboning machine 44 as appropriate.

The refuse hopper 46 typically stores inorganic substrate and refuse materials discharged from the plant awaiting transportation off site.

Stream 87 is the stream of organic materials directed into the process. Water may be added to the organic material as desired to create a pumpable slurry. The organic material slurry pump 38 is provided to transport the organic material slurry into the process. Stream 88 is the stream of organic material slurry directed into the primary digestion reactor unit 36. The primary digestion reactor unit 36 digests the organic materials, such as by the three stage process described with respect to FIG. 4. Stream 127 is the stream of the products of anaerobic digestion, which preferably are substantially methane and carbon dioxide gases saturated with water vapor. The gas cooler and gas dryer 12 is provided to control the entry of water vapor entrained in the mixed gas stream directed into the subsequent gas reformer 106. The presence of water in the mixed gas stream will:
1. consume energy provided for gas reforming,
2. permit the process of steam methane reforming to occur reducing the ability of the subsequent gas reformer to reform the carbon monoxide into desired products,
3. cause metallurgical problems in the process modules and piping.

In this preferred embodiment, the uses of steam or condensate include without limitation:
1. Building heating,
2. Reactor and tank heating within the plant,
3. Greenhouse heating,
4. Aquaculture heating,
5. Preparation of boiler feed water (BFW) and media,
6. Thawing of received materials,
7. Steam explosion of woody material in preparation for use as a feed material for the plant.

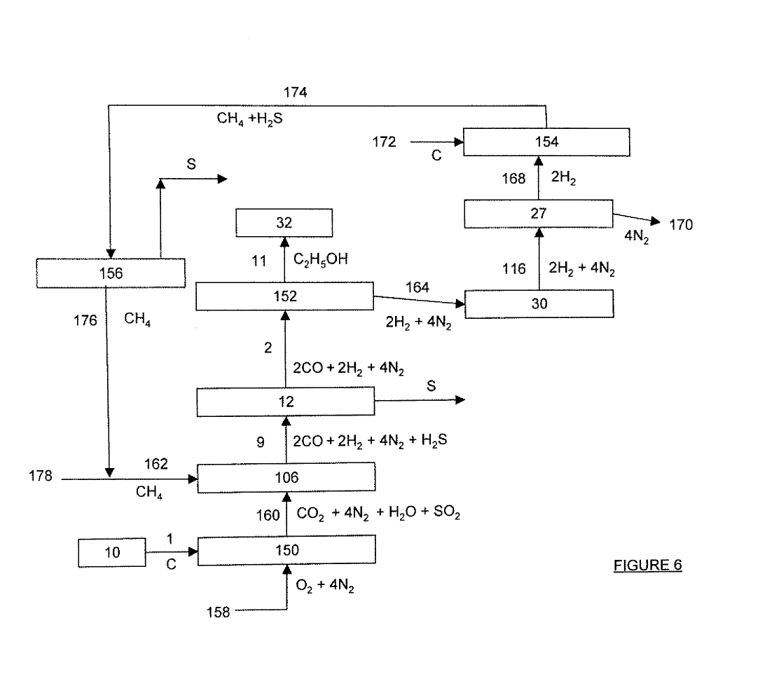

The sixth preferred embodiment, as exemplified in FIG. 6, exemplifies the use of the process to convert coal to, erg., ethanol. As shown therein, the process comprises a combustion unit 150, multi-stage gas reforming unit 106, gas cleaning unit 12, alcohol fermentation plant 152, gas compression unit 30, gas separation unit 27, carbonization unit 154 and a second gas cleaning unit 156.

A feed stream 1 of carbonaceous or hydrocarbonaceous material, including, without limitation, coal, vegetable material or oil from a feed source 10 is prepared by means known in the art and introduced into the combustion unit 150 where it is burned in a feed stream 158 oft e.g., combustion air or oxygen. For the purpose of explaining this alternate embodiment, coal will be used for this discussion. The coal burns to produce a combustion products air stream 160 comprising carbon dioxide with small quantities of water and impurities such as sulfur dioxide, metallic oxides and ash forming, as well as the presence of nitrogen if the combustion gas stream 158 included nitrogen, and evolving high quality heat. A feed stream of methane, stream 162, is preferably prepared and introduced into the multi stage gas reformer unit 106 where it is combined with the carbon dioxide from combustion products stream 160.

A mixture, which preferably is of approximately equal parts of methane and carbon dioxide gases, is reformed to produce a synthesis gas stream 9, which comprises a mixture of carbon monoxide and hydrogen with minor amounts of methane and carbon dioxide and nitrogen if present in the combustion gas stream 158.

Preferably, the gas cleaning unit 12 cleans the gas, removing sulfur and its compounds and other impurities forming cleaned synthesis gas stream 2. The sulfur may be removed as hydrogen sulfide as taught in Shessel et al. (U.S. Pat. No. 5,690,482). The mixed carbon monoxide and hydrogen, with the nitrogen, if used, stream 2, is used as a substrate for the growth and support of the microbial catalyst in the alcohol fermentation plant 152. Similar to the preferred embodiment of FIG. 4 and as described in the forgoing, the alcohol fermentation plant 152 is preferably composed of the alcohol fermentation reactor, higher hydrocarbon compounds digestion reactor and digestion reactor and associated equipment and connecting streams. The alcohol fermentation plant 152 converts the carbon monoxide portion of the feed stream of mixed carbon monoxide and hydrogen stream 2, to alcohol or mixed alcohols and other higher hydrocarbon compounds forming product stream 11 which may be stored in storage unit 32.

In the operation of the alcohol fermentation reactor the carbon monoxide is consumed in the process but that the hydrogen is passed through the process forming, with the nitrogen, if used, gas stream 164, see for example Gaddy (U.S. Pat. No. 5,173,429). The gas stream 2 is preferably recycled repeatedly through the pH controlled aqueous media providing additional opportunities the clean the gas.

The gas compression unit 30 is optionally provided to raise the pressure of the mixture of hydrogen and of nitrogen, if used, stream 164, in preparation for subsequent gas separation and conversion processes forming pressurized gas stream 116. The gas separation unit 27 is optionally provided to separate the hydrogen and nitrogen, if used, gases as required for further processes. Gas separation may be utilized to:
1) separate at least a portion of the hydrogen from the mixture of hydrogen and nitrogen, if used, forming hydrogen rich stream 168, to be used in the carbonization unit 154,
2) separate at least a portion of the nitrogen from the mixture of hydrogen and nitrogen, if used, forming nitrogen rich stream 170 for venting to the atmosphere.

A feed stream of coal is preferably prepared, stream 172, and introduced into the carbonization unit 154 where it is combined with the hydrogen in stream 168. Any process known in the art may be used. Preferably, a process such as that disclosed in Albright et al. (U.S. Pat. No. 4,002,535) is utilized. The carbonization unit 154 anaerobically converts the coal and hydrogen to methane with small quantities of impurities such as hydrogen sulfide, metallic compounds and ash forming product stream 174. The second gas cleaning unit 156 may optionally be provided to clean the gas, removing sulfur and its compounds and other impurities forming cleaned gas stream 176 or the impurities may remain in the process gas streams to be removed in gas cleaning unit 12.

The methane is then preferably combined with a quantity of make up methane 178 to restore the stoichiometric balance of carbon dioxide and methane required in gas reforming plant 106. In this fashion any carbonaceous or hydrocarbon material, particularly sulfur bearing materials, may be converted to alcohol or mixed alcohols and other higher hydrocarbon compounds. Due to the energy positive nature of the process of the instant invention, it is suitable for addition to or substitution for existing power boilers and similar equipment as a method for reducing or eliminating sulfur pollution in the environment by converting low cost sulfur bearing coal and heavy high sulfur oils to alcohols prior to firing in a fuel burning appliance.

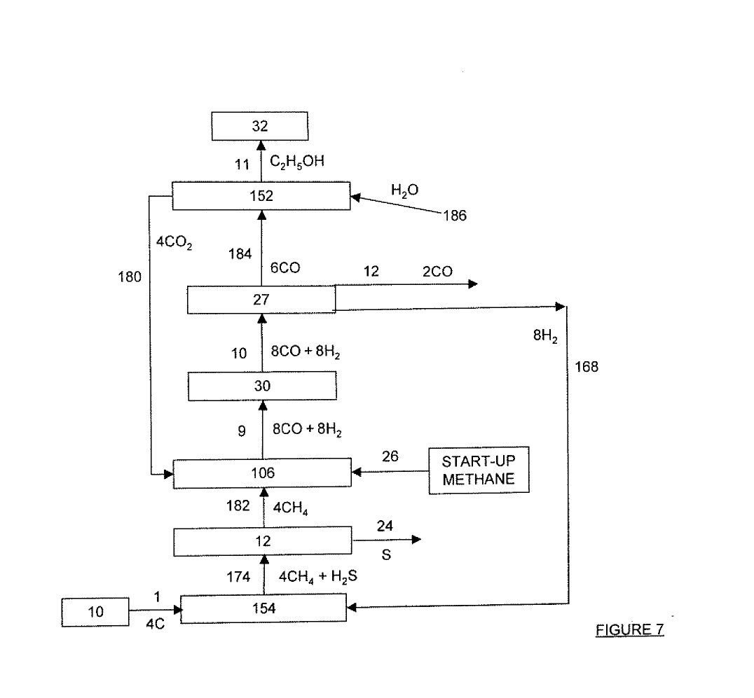

The seventh preferred embodiment, as exemplified in FIG. 7, exemplifies an alternate use of the process to convert coal to, e.g., ethanol the process comprises an alcohol fermentation plant 152, gas cleaning unit 12, multi-stage gas reforming unit 106, gas compression unit 30, gas separation unit 27 and carbonization unit 154. The alcohol fermentation plant 152 is preferably composed of the alcohol fermentation reactor, higher hydrocarbon compounds digestion reactor and digestion reactor and associated equipment and connecting streams as exemplified in FIG. 4.

Gaddy (U.S. Pat. No. 5,593,886) discloses that microbial catalysts ferment carbon monoxide according to the process:
6CO+3H₂O→C₂H₅OH+4CO₂.

By importing hydrogen the process:
2CO₂+6H₂→C₂H₅OH+3H₂O

may be added. Therefore, the conversion rate of carbon monoxide feed to ethanol product may be up to 33⅓%. By importing hydrogen, the conversion rate of carbon feed to ethanol product may be increased up to 66⅔% but half of the imported hydrogen is converted to water making it unavailable for any useful purpose. By applying the principles of the instant process, the conversion rate is increased to 50% without the need of foregoing such a valuable resource as hydrogen.

Beginning with the process:
6CO+3H₂O→C₂H₅OH+4CO₂, (1)
in the alcohol fermentation plant 152 the carbon monoxide, stream 184 and water via stream 186, is converted to, e.g., ethanol forming product stream 11 and carbon dioxide stream 180. The carbon dioxide stream 180, is fed to the multi-stage gas reforming unit 106 where it is mixed, preferably, with an equal quantity of methane available via stream 182. Similar to the preferred embodiment of FIG. 1 and as described in the forgoing, the mixture of approximately equal parts of methane and carbon dioxide gases combined from streams 180 and 182 is preferably progressively heated, reformed to a mixture of carbon monoxide and hydrogen with minor amounts of methane and carbon dioxide and cooled by a series of gas reforming steps in the multi-stage gas reforming unit 106 using heat energy from a variety of energy sources forming synthesis gas stream 9. Reforming permits the following process to proceed:
CH₄+CO₂→2CO+2H₂ (2)

The gas compression unit 30 raises the pressure of the mixture of hydrogen and carbon monoxide, synthesis gas stream 9, to prepare pressurized synthesis gas stream 10 in preparation for subsequent gas separation and conversion processes. The gas separation unit 27 preferably separates the hydrogen and carbon monoxide gases in stream 10 as required for further processes. Gas separation may be utilized to:
1. separate at least a portion of the hydrogen from the mixture of hydrogen and carbon monoxide forming hydrogen rich stream 168 for use in the carbonization unit 154,
2. separate at least a portion of the carbon monoxide from the mixture of hydrogen and carbon monoxide for use as a substrate for the growth and support of the microbial catalyst in the alcohol fermentation plant, 152 forming pressurized carbon monoxide rich stream 184.

A feed stream of carbonaceous or hydrocarbonaceous material, including, without limitation, coal, organic animal or vegetable material or oil, feed stream 1, is prepared and introduced into the carbonization unit 154 where it is combined with the feed stream of hydrogen, stream 168. Coal will be used for this discussion. The carbonization unit 154 anaerobically converts the coal and hydrogen to methane with small quantities of impurities such as hydrogen sulfide, metallic compounds and ash forming stream 174 according to the following process:
4C+8H₂→4CH₄ (3)

The gas cleaning unit 12 is provided to clean the methane gas, removing sulfur and its compounds and other impurities forming stream 9. In this fashion any carbonaceous or hydrocarbon material may be converted to alcohol or mixed alcohols and other higher hydrocarbon compounds. Adding equations 1, 2 and 3:
4C+3H₂O→C₂H₅OH+2CO,
demonstrating that the efficiency of the conversion of carbon to ethanol has been improved without the requirement to import hydrogen. Because of the ability of the process to remove sulfur and sulfur compounds from carbonaceous materials, it is particularly well suited for use in upgrading high sulfur fuels.

Further embodiments of the instant process are possible through further combinations of the foregoing elements of the instant process.

The terms and expressions which have been employed herein are used as terms of description and not of limitation and there is no intention in the use of such terms and expressions of excluding any equivalents of the features shown and described or portions thereof but it is recognized that various modifications are possible within the scope of the invention claimed.

1. A process for the production of a hydrocarbon comprising the steps of:
(a) providing a gaseous feed stream comprising carbon dioxide and a source of hydrogen;
(b) subjecting the feed stream to elevated temperatures in an oxygen reduced environment and obtaining a synthesis gas stream comprising carbon monoxide and hydrogen gas; and,
(c) utilizing the synthesis gas stream to produce at least one hydrocarbon product.

2. The process of claim 1 wherein the hydrogen is obtained from a hydrocarbon.

3. The process of claim 2 wherein the hydrocarbon comprises methane.

4. The process of claim 2 wherein the feed stream comprises approximately amounts of methane and carbon dioxide on a mole basis.

5. The process of claim 1 wherein the hydrocarbon product comprises at least one alcohol.

6. The process of claim 3 wherein the at least one alcohol comprises ethanol.

7. The process of claim 1 wherein the carbon dioxide is obtained from one or more of biogas, landfill gas, alcohol production, and the combustion of hydrocarbons.

8. The process of claim 1 wherein the elevated temperature is from 1,100° C.-3,200° C.

9. The process of claim 1 wherein the elevated temperature is from 1,700° C.-2,300° C.

10. The process of claim 1 wherein the elevated temperature is about 2,200° C.

11. The process of claim 1 wherein the oxygen reduced environment comprises less than 10 weight percent oxygen gas.

12. The process of claim 1 wherein step (b) is conducted in the absence of the addition of combustion air or oxygen.

13. The process of claim 1 wherein the oxygen reduced environment comprises essentially no oxygen gas.

14. The process of claim 1 wherein step (b) comprises a dry reforming process.

15. The process of claim 1 wherein step (b) comprises a multi-stage dry reforming process.

16. The process of claim 14 further comprising utilizing combustion of a fuel to produce electricity and hot combustion gasses and utilizing at least one of a portion of the electricity and at least a portion of the hot combustion gasses to provide heat to the dry reforming step.

17. The process of claim 14 further comprising utilizing excess heat available in a plant to provide heat to the dry reforming step and to produce steam and utilizing at least a portion of the steam to produce electricity.

18. The process of claim 16 further comprising utilizing excess heat available in a plant to provide heat to the dry reforming step and to produce steam and utilizing at least a portion of the steam to produce electricity.

19. The process of claim 1 further comprising treating coal to obtain carbon dioxide, which is utilized to produce the feed gas.

20. The process of claim 19 further comprising treating coal to obtain methane which is utilized to produce carbon dioxide.

21. The process of claim 3 further comprising treating coal to obtain methane.
